(19) United States
(12) Patent Application Publication
(10) Pub. No.: US 2022/0257578 A1
(43) Pub. Date: Aug. 18, 2022

(54) COMPOSITION AND METHOD FOR TREATING CANDIDA AURIS INFECTION

(71) Applicant: Appili Therapeutics Inc., Halifax (CA)

(72) Inventors: Thomas F. PATTERSON, San Antonio, TX (US); Nathan P. WIEDERHOLD, San Antonio, TX (US); Laura K. NAJVAR, San Antonio, TX (US)

(73) Assignee: Appili Therapeutics Inc., Halifax (CA)

(21) Appl. No.: 17/735,144

(22) Filed: May 3, 2022

Related U.S. Application Data
(63) Continuation of application No. PCT/JP2020/040374, filed on Oct. 28, 2020.
(60) Provisional application No. 62/930,076, filed on Nov. 4, 2019.

(51) Int. Cl. A61K 31/445; A61P 31/10

(52) U.S. Cl. CPC A61K 31/445; A61P 31/10

(57) ABSTRACT

Candida auris infection in a subject is treated with 4-{3-[4-(3-{4-[amino(imino)methyl]phenoxy}propyl)-1-piperidinyl]propoxy}benzamidine (T-2307) or a pharmaceutically acceptable salt thereof.

### CROSS-REFERENCE TO RELATED APPLICATIONS

This application is a continuation of International Application No. PCT/JP2020/040374 filed on Oct. 28, 2020, which claims benefit of U.S. Provisional Application No. 62/930,076 filed on Nov. 4, 2019, the entire contents of which are incorporated herein by reference.

### FIELD

This application relates to compositions and methods for treating an infectious disease in a subject, particularly for treating Candida auris infection.

### BACKGROUND

Candida auris is an emerging pathogen that has now been detected in institutions on multiple continents. Invasive infections caused by this species of fungus are associated with high mortality rates up to 59%. T-2307 (see Scheme 1) is an arylamidine and causes the collapse of fungal mitochondrial membrane potential. This agent has been shown to have potent in vitro and in vivo activity against some Candida species (e.g. Candida albicans). Unfortunately, C. auris is notoriously difficult to treat, C. auris isolates often being resistant to multiple antifungals, including fluconazole and other azoles, and up to one third of isolates may be resistant to amphotericin B, with echinocandin resistance as well. Therefore, even though T-2307 has been effective against some Candida species, there is no a priori expectation that T-2307 would exhibit efficacy in treating C. auris.

There remains a need for an effective treatment for C. auris infections.

### SUMMARY

In one aspect, there is provided a method of treating Candida auris infection in a subject comprising administering to the subject infected with Candida auris a pharmaceutical composition comprising: a therapeutically effective amount of 4-{3-[4-(3-{4-[amino(imino)methyl]phenoxy}propyl)-1-piperidinyl]propoxy}benzamidine (T-2307) or a pharmaceutically acceptable salt thereof; and, a pharmaceutically acceptable carrier, diluent and/or excipient.

In another aspect, there is provided a use of a pharmaceutical composition comprising: a therapeutically effective amount of 4-{3-[4-(3-{4-[amino(imino)methyl]phenoxy}propyl)-1-piperidinyl]propoxy}benzamidine (T-2307) or a pharmaceutically acceptable salt thereof; and, a pharmaceutically acceptable carrier, diluent and/or excipient, for treating Candida auris infection in a subject.

It has now been surprisingly found that T-2307 is effective in treating Candida auris infections, and is particularly at specified dosage range. Compared to controls, T-2307 significantly improves survival rates of subjects infected with C. auris.

Further features will be described or will become apparent in the course of the following detailed description. It should be understood that each feature described herein may be utilized in any combination with any one or more of the other described features, and that each feature does not necessarily rely on the presence of another feature except where evident to one of skill in the art.

### BRIEF DESCRIPTION OF THE DRAWINGS

For clearer understanding, preferred embodiments will now be described in detail by way of example, with reference to the accompanying drawings, in which:

FIG. 1 depicts survival curves in mice inoculated intravenously with C. auris and treated with vehicle control, fluconazole 20 mg/kg PO QD, or caspofungin 10 mg/kg IP QD (A) or T-2307 at doses of 0.75 mg/kg, 1.5 mg/kg, or 3 mg/kg SC QD (B). Treatment started one day post-inoculation and continued for 7 days. Mice were then followed off therapy until day 21 post-inoculation (14 days after therapy stopped). Black square—vehicle control; white circle—fluconazole 20 mg/kg; black circle—caspofungin 10 mg/kg; inverted gray triangle T-2307 0.75 mg/kg; gray rectangle—T-2307 1.5 mg/kg; gray triangle—T-2307 3 mg/kg. N=10 mice per group.

FIG. 2 depicts graphs showing kidney (A) and brain (B) fungal burden (CFU/g) in mice with invasive candidiasis secondary to C. auris in the fungal burden arm. Colony-forming units were measured on day 8 post-inoculation after 7 days of therapy. N=10 mice in the vehicle control and treatment groups; N=5 mice in the 24-hour control group.

FIG. 3 depicts graphs showing kidney (A) and brain (B) fungal burden (CFU/g) in mice with invasive candidiasis secondary to C. auris in the survival arm. Colony-forming units were measured on day 8 post-inoculation after 7 days of therapy. N=10 mice in the vehicle control and treatment groups; N=5 mice in the 24-hour control group. Black circles represent mice that succumbed to infection prior to day 21; gray circles represent mice that survived to the survival endpoint.

### DETAILED DESCRIPTION

The compound 4-{3-[4-(3-{4-[amino(imino)methyl]phenoxy}propyl)-1-piperidinyl]propoxy}benzamidine, also known as T-2307, is an arylamidine having a molecular formula of C₂₅H₃₅N₅O₂ and a free-base structure as shown in Scheme 1.

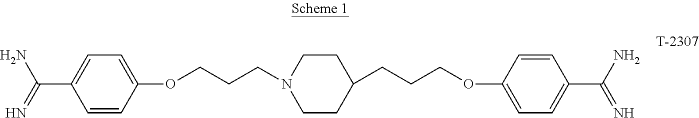 [SMILES: N=C(N)c1ccc(OCCCC2CCN(CCCOc3ccc(C(=N)N)cc3)CC2)cc1]

Examples of pharmaceutically acceptable salts of T-2307 include: salts with mineral acids (e.g. hydrochloric acid, hydrobromic acid, nitric acid, phosphoric acid or sulfuric acid); salts with carboxylic acids (e.g. formic acid, acetic acid, citric acid, oxalic acid, fumaric acid, lactic acid, maleic acid, succinic acid, malic acid, tartaric acid, aspartic acid, trichloroacetic acid, trifluoroacetic acid, oleic acid or caprylic acid); and salts with sulfonic acids (e.g. methanesulfonic acid, benzene sulfonic acid, p-toluene sulfonic acid, mesitylene sulfonic acid or naphthalene sulfonic acid). Salts with hydrochloric acid, lactic acid, methanesulfonic acid or acetic acid are preferable, and a salt with hydrochloric acid is more preferable. The salt with hydrochloric acid is preferably a dihydrochloride or trihydrochloride, more preferably a trihydrochloride. The T-2307 or pharmaceutically acceptable salt thereof may be in the form of a hydrate or solvate, preferably a monohydrate or pentahydrate, more preferably a pentahydrate. A trihydrochloride salt pentahydrate of T-2307 (C₂₅H₃₅N₅O₂.3HCl.5H₂O) is particularly preferred.

T-2307 and pharmaceutically acceptable salts thereof can be produced by conventional methods. For example, T-2307 and pharmaceutically acceptable salts thereof can be produced by the methods described in, for example, U.S. Pat. No. 7,700,623 issued Apr. 20, 2010, the entire contents of which is herein incorporated by reference.

The subject is preferably a human or an animal. The animal may be a mammal or a bird. Some examples of animals are mice, rats, guinea pigs, rabbits, pigs, horses, cows, dogs, cats, monkeys, chickens and turkeys. The Candida auris infection may be a disseminated and/or invasive infection. The Candida auris infection may be a kidney or brain infection.

The T-2307 may be administered to the subject by any suitable route, for example orally, parenterally or topically. Oral and parenteral administration are preferred. Parenteral administration may be by intramuscular, subcutaneous, intravenous or intradermal injection, preferably subcutaneous or intravenous injection.

The T-2307 or pharmaceutically acceptable salt thereof may be prepared in a pharmaceutical composition. In some embodiments, the pharmaceutical composition includes T-2307 or pharmaceutically acceptable salt thereof and a pharmaceutically acceptable carrier, diluent and/or excipient. Formulation of the composition may be achieved in a variety of ways that are known in the art. A summary of formulation techniques is found in Remington: The Science and Practice of Pharmacy, 22nd Edition, Lippincott Williams & Wilkins, (2012); and Encyclopedia of Pharmaceutical Technology, eds. J. Swarbrick and J. C. Boylan, 2006, Marcel Dekker, New York, each of which is incorporated herein by reference.

Pharmaceutically acceptable carriers, diluents and/or excipients in the pharmaceutical compositions are nontoxic to recipients at the dosages and concentrations employed. Acceptable carriers, diluents and/or excipients are generally known in the art and may include, for example: inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); lubricating agents, glidants, and antiadhesives (e.g., magnesium stearate, zinc stearate, stearic acid, silicas, hydrogenated vegetable oils, or talc); colorants; flavoring agents; plasticizers; humectants; buffering agents; and the like.

The pharmaceutical compositions can be prepared in a variety of dosage forms, for example, intravenous dosage forms, subcutaneous dosage forms and oral dosage forms (e.g., ingestible solutions, drug release capsules). Some specific examples of dosage forms are tablets, capsules, pills, powders, granulates, suspensions, emulsions, solutions, gels including hydrogels, pastes, ointments, creams, plasters, drenches, syrups, dusts, eye drops, nasal drops, eardrops, patches, osmotic delivery devices, suppositories, enemas, injectables, implants, sprays, preparations suitable for iontophoretic delivery, or aerosols. The compositions may be formulated according to conventional pharmaceutical practice.

The pharmaceutical composition is preferably administered parenterally in the form of an injectable formulation.

The administration method, the dose and the number of doses of the composition can be appropriately selected according to the age, body weight and symptoms of the subject. The pharmaceutical composition may be formed in a unit dose form as needed. Usually, the composition may be administered to an adult once to several times a day, for example once, twice, thrice or more per day), preferably through an oral or parenteral (e.g., by injection, by drip infusion or through a rectal site) route.

The composition may be packaged in a commercial package together with instructions from use of the composition to treat C. auris infection. Commercial packages may include ampoules, blister packs, bottles, jars and the like.

The amount of T-2307 or pharmaceutically acceptable salt thereof included in the pharmaceutical composition is such that a suitable daily dose within the designated range is provided to the subject. For example, for humans a dosing regimen that achieves a total daily exposure in a range of 0.01-1000 mg/kg of body weight, based on weight of the T-2307 free base, is suitable. Suitable conversion of the daily doses for pharmaceutically acceptable salts of T-2307 can be readily determined when required.

Converting the values reported in the Examples below in mice to human equivalent dosing based on body surface area (Guidance for Industry Estimating the Maximum Safe Starting Dose in Initial Clinical Trials for Therapeutics in Adult Healthy Volunteers. U.S. Department of Health and Human Services Food and Drug Administration Center for Drug Evaluation and Research (CDER), July 2005. Available online at https://www.fda.gov/media/72309/download, the entire contents of which is herein incorporated by reference), for humans, a dosing regimen that achieves a total daily exposure, based on weight of the T-2307 free base, may be in the range of at least 0.10 mg/kg of body weight, or at least 0.20 mg/kg of body weight, or at least 0.21 mg/kg of body weight, or at least 0.22 mg/kg of body weight, or at least 0.23 mg/kg of body weight, or at least 0.24 mg/kg of body weight, or at least 0.25 mg/kg of body weight, or at least 0.3 mg/kg of body weight, or at least 0.5 mg/kg of body weight, or at least 0.75 mg/kg of body weight, or at least 1 mg/kg of body weight, or at least 2 mg/kg of body weight, or at least 3 mg/kg of body weight. For a human, the total daily exposure may be no more than 8 mg/kg of body weight, or no more than 7 mg/kg of body weight, or no more than 6 mg/kg of body weight, or no more than 5 mg/kg of body weight, or no more than 4 mg/kg of body weight. Preferred total daily exposures may be in the range of from 0.1 to 8 mg/kg of body weight, or from 0.2 to 2 mg/kg of body weight, or from 0.2 to 1 mg/kg of body weight, or from 0.21 to 0.5 mg/kg of body weight, or from 0.24 to 0.35 mg/kg of body weight.

The composition preferably exhibits a minimum inhibitory concentration (MIC) to 50% inhibition in vitro against an infecting isolate of C. auris of 0.2 μg/mL or lower, or 0.1 μg/mL or lower, or 0.05 μg/mL or lower, or 0.025 μg/mL or lower, or 0.02 μg/mL or lower, or 0.015 μg/mL or lower.

The T-2307 or pharmaceutically acceptable salt thereof can also be administered simultaneously, separately, or in a specific order, with other drugs including an azole antifungal agent, a polyene antifungal agent, a candin antifungal agent, a fluoropyrimidine antifungal agent, and immunosuppressant or any mixture thereof.

### EXAMPLES

Methods:

In vitro susceptibility testing was performed according to the CLSI M27-A3 standard, the contents of which are herein incorporated by reference, against 10 isolates available from the U.S. Food and Drug Administration Center for Disease Control Antibiotic Resistance (FDA CDC AR) Isolate Bank and 13 clinical isolates that were received for testing by the Fungus Testing Laboratory at University of Texas Health Science Center at San Antonio (UTHSCSA). The minimum inhibitory concentration (MIC) of T-2307 was measured as the lowest concentration that inhibited both 50% and 100% of growth compared to the drug-free control after 24 hours of incubation at 35° C., while the MICs of fluconazole and caspofungin were measured at 50% growth inhibition.

Male Institute of Cancer Research (ICR) mice were rendered neutropenic with a single dose of 5-fluorouracil (5 mg/mouse) administered 24 hours prior to inoculation, and a clinical isolate of C. auris (DI 17-46) was used to infect mice via the lateral tail vein as previously described (Wiederhold et al. 2019. Antimicrob Agents Chemother. 63:e02233-18, the entire contents of which is herein incorporated by reference). Treatment with vehicle control, T-2307 (0.75, 1.5, or 3 mg/kg subcutaneously (SC) once daily), fluconazole (20 mg/kg per orally (PO) once daily), or caspofungin (10 mg/kg intraperitoneally (IP) once daily) began 1-day post-inoculation and continued for 7 days. In the fungal burden arm, mice were humanely euthanized on day 8 post-inoculation, and kidneys and brains were collected, weighed, and homogenized for analysis of colony-forming units (CFU/g). In the survival arm, mice were followed off therapy for 14 days, until day 21 post-inoculation. Fungal burden was also assessed in the survival arm on day 21 or on the day the mice succumbed to infection. Differences in survival were assessed by Kaplan-Meier analysis with the log-rank test. ANOVA with Tukey's post-test for multiple comparisons were used to assess for differences in fungal burden and geometric mean (GM) MIC values.

Results:

As seen in Table 1, T-2307 demonstrated potent in vitro activity against C. auris. The MIC range using the 50% inhibition endpoint was ≤0.008 μg/mL to 0.015 μg/mL, but was markedly higher when measured using the 100% inhibition endpoint (0.25 μg/mL to >4 μg/mL). Overall, the MICs for T-2307 using the 50% inhibition endpoint were lower than those for fluconazole (range 0.5 μg/mL to >64 μg/mL) and caspofungin (0.015 μg to >8 μg/mL), and the GM MIC for T-2307 (0.011 μg/mL) was significantly lower than that observed for both fluconazole and caspofungin (14.6 μg/mL and 0.24 μg/mL, respectively; p<0.0001). The MICs for T-2307, fluconazole, and caspofungin against the infecting isolate were <0.008 μg/mL, >64 μg/mL, and 0.25 μg/mL, respectively.

| Antifungal | T-2307 | | Fluconazole | Caspofungin |
|---|---|---|---|---|
| % Inhibition | 50% | 100% | 50% | 50% |
| MIC Range | ≤0.008-0.015 | 0.125 -> 4 | 0.5 -> 64 | ≤0.015 -> 8 |
| MIC50 | 0.015 | >4 | 4 | 0.25 |
| MIC90 | 0.015 | >4 | >64 | 0.5 |
| GM MIC | 0.011 | 2.189 | 14.6 | 0.24 |

In vitro activity of T-2307, fluconazole, and caspofungin against C. auris. Minimum inhibitory concentrations (MICs; μg/mL) read for T-2307 (50% and 100% inhibition), fluconazole (50% inhibition), and caspofungin (50% inhibition) after 24 hours of incubation at 35° C. MIC50 and MIC90 are lowest concentrations that inhibited 50% and 90%, respectively, of the isolates tested. GM MIC is geometric mean MIC.

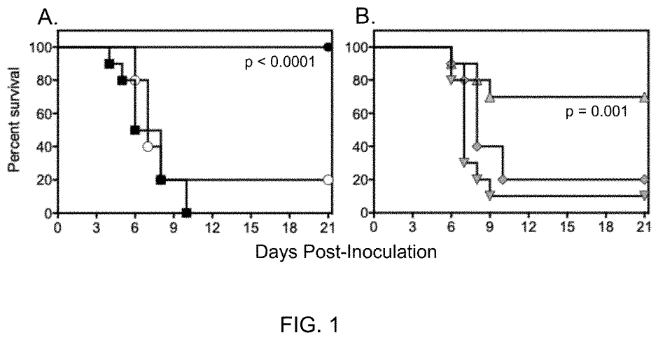

The in vitro activity of T-2307 did translate into in vivo efficacy, as the highest dose of T-2307 (3 mg/kg) resulted in significant improvements in median and percent survival (>21 days and 70%, respectively) compared to control (5 days and 0%; p<0.01) (FIG. 1). Similar improvements in survival were also observed in mice treated with high dose caspofungin (>21 days and 100%; p<0.001). In contrast, the lower doses of T-2307 and fluconazole did not improve survival.

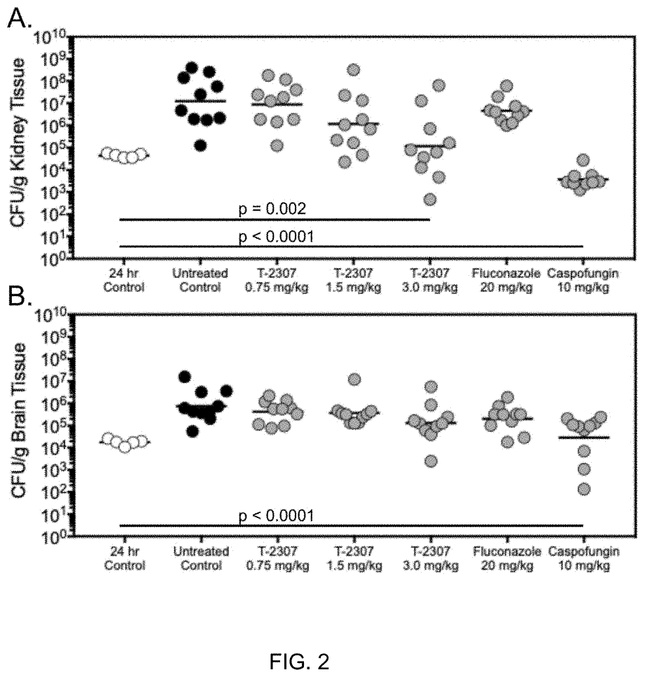

In the fungal burden arm, significant reductions in kidney CFUs were also observed in mice treated with T-2307 3 mg/kg (mean 5.06 log₁₀ CFU/g) and caspofungin (3.21 log₁₀ CFU/g) compared to control (7.09 log₁₀ CFU/g; p<0.01) (FIG. 2). The activity of T-2307 was static in nature, as the fungal burden in the 3 mg/kg group was similar to that observed in the 24-hour group measured just prior to the start of therapy. Reductions in kidney fungal burden were not observed in mice treated with the lower doses of T-2307 or fluconazole. Brain fungal burden observed in the caspofungin group (4.45 log₁₀ CFU/g) was significantly lower than control (5.88 log₁₀ CFU/g; p<0.001) on day 8 post-inoculation but not in mice treated with T-2307 or fluconazole.

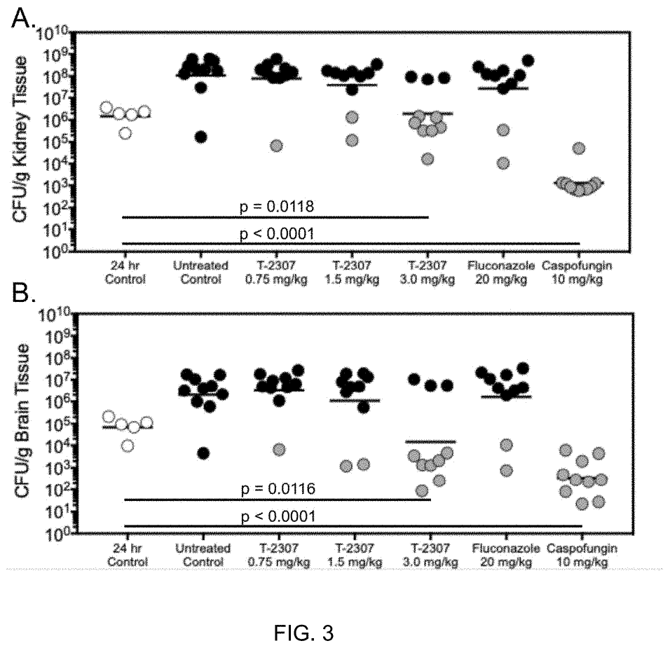

In the survival arm kidney, fungal burden was significantly lower in the T-2307 3 mg/kg (6.28 log₁₀ CFU/g) and caspofungin groups (3.11 log₁₀ CFU/g) compared to control (8.04 log₁₀ CFU/g; p<0.01) (FIG. 3). Interestingly, brain fungal burden was significantly reduced in mice treated with T-2307 3 mg/kg (4.16 log₁₀ CFU/g) and caspofungin (2.51 log₁₀ CFU/g) versus control (6.31 log₁₀ CFU/g; p<0.01). Previous studies have demonstrated reductions in brain and ocular tissue fungal burden in mice infected with Cryptococcus gattii and Candida albicans, respectively, and treated with T-2307. In the current study, there was also a clear relationship between fungal burden and survival, as treated mice that survived to the day 21 endpoint had lower kidney and brain fungal burden compared to those that succumbed to infection.

These results demonstrate that T-2307 is effective against invasive infections caused by C. auris, as both in vitro and in vivo activity were observed against this emerging pathogen. The reductions in fungal burden observed in this study were less than those previously observed against echinocandin resistant C. albicans in immunocompetent mice, but were similar to those observed against echinocandin resistant C. glabrata infections in neutropenic mice. This suggests that the in vivo efficacy of T-2307 may be influenced by immune status, similar to what has been reported for other antifungals in murine models of infection.

The novel features will become apparent to those of skill in the art upon examination of the description. It should be understood, however, that the scope of the claims should not be limited by the embodiments, but should be given the broadest interpretation consistent with the wording of the claims and the specification as a whole.

1. A method of treating Candida auris infection in a subject comprising administering to the subject infected with Candida auris a pharmaceutical composition comprising:
a therapeutically effective amount of 4-{3-[4-(3-{4-[amino(imino)methyl]phenoxy}propyl)-1-piperidinyl]propoxy}benzamidine (T-2307) or a pharmaceutically acceptable salt thereof; and,
a pharmaceutically acceptable carrier, diluent and/or excipient.

2. The method of claim 1, wherein the composition is administered to the subject using a dosing regimen that achieves a total daily exposure of the T-2307 of 0.01-1000 10 mg/kg of body weight, based on weight of T-2307 free base.

3. The method of claim 1, wherein the composition is administered to the subject using a dosing regimen that achieves a total daily exposure of the T-2307 of at least 0.20 mg/kg of body weight, based on weight of T-2307 free base.

4. The method of claim 1, wherein the composition is administered to the subject using a dosing regimen that achieves a total daily exposure of the T-2307 of at least 0.24 mg/kg of body weight, based on weight of T-2307 free base.

5. The method of claim 1, wherein the composition is administered to the subject using a dosing regimen that achieves a total daily exposure of the T-2307 of at least 3 mg/kg of body weight, based on weight of T-2307 free base.

6. The method of claim 1, wherein the composition is administered to the subject using a dosing regimen that achieves a total daily exposure of the T-2307 of no more than 8 mg/kg of body weight, based on weight of T-2307 free base.

7. The method of claim 1, wherein the infection is a kidney or brain infection.

8. The method of claim 1, wherein the subject is a mammal.

9. The method of claim 1, wherein the subject is human.

10. Use of a pharmaceutical composition comprising: a therapeutically effective amount of 4-{3-[4-(3-{4-[amino(imino)methyl]phenoxy}propyl)-1-piperidinyl]propoxy}benzamidine (T-2307) or a pharmaceutically acceptable salt thereof; and, a pharmaceutically acceptable carrier, diluent and/or excipient, for treating Candida auris infection in a subject.

11. The use of claim 10, wherein the composition is provided to the subject using a dosing regimen that achieves a total daily exposure of the T-2307 of 0.01-1000 mg/kg of body weight, based on weight of T-2307 free base.

12. The use of claim 10, wherein the composition is provided to the subject using a dosing regimen that achieves a total daily exposure of the T-2307 of at least 0.20 mg/kg of body weight, based on weight of T-2307 free base.

13. The use of claim 10, wherein the composition is provided to the subject using a dosing regimen that achieves a total daily exposure of the T-2307 of at least 0.24 mg/kg of body weight, based on weight of T-2307 free base.

14. The use of claim 10, wherein the composition is provided to the subject using a dosing regimen that achieves a total daily exposure of the T-2307 of at least 3 mg/kg of body weight, based on weight of T-2307 free base.

15. The use of claim 10, wherein the composition is provided to the subject using a dosing regimen that achieves a total daily exposure of the T-2307 of no more than 8 mg/kg of body weight, based on weight of T-2307 free base.

16. The use of claim 10, wherein the infection is a kidney or brain infection.

17. The use of claim 10, wherein the subject is a mammal.

18. The use of claim 10, wherein the subject is human.
